(19) United States
(12) Patent Application Publication
(10) Pub. No.: US 2007/0106375 A1
(43) Pub. Date: May 10, 2007

(54) Bifurcated stent assembly

(76) Inventor: Carlos Vonderwalde, Richmond (CA)

Correspondence Address: Martin Moynihan;PRTSI, Inc., P.O. Box 16446, Arlington, VA, 22215, US

(21) Appl. No.: 11/267,311

(22) Filed: Nov. 7, 2005

(51) Int. Cl. A61F 2/06

(52) U.S. Cl. 623/1.35

(57) ABSTRACT

A bifurcated stent assembly using a flexible membrane, preferably a tubular membrane, as a stent connector to bendably hold and functionally associate two or more component stents is disclosed. The flexible stent connector allows the relative orientation of the two stents to be varied with no deformation of the component stents. In preferred embodiments, the stent connector is configured to provide support in the vicinity of the bifurcation point of a vessel in which deployed.

### FIELD AND BACKGROUND OF THE INVENTION

The present invention relates to the field of surgery and more particularly, to a method and a device useful for maintaining patency of a bifurcated lumen, especially of the cardiovascular system.

A stent is a device deployed inside a lumen of a bodily vessel to physically maintain patency of the vessel. Typical vessels treated with stents include respiratory ducts, gastrointestinal ducts, lymphatic ducts, blood vessels and especially arteries that are occluded, stenosed, aneuritic, physically damaged, diseased, collapsing or weakened.

Stents are usually outwardly radially expandable, having a substantially tubular shape both in an unexpanded state with a small radial dimension and in any one of the expanded states with larger radial dimensions. Various constructions of stents are known including rolled-up sheets, slotted or otherwise cut-out tubes and bent wires.

For deployment inside a lumen of a bodily vessel an expandable stent is placed in an unexpanded state on a deployment catheter, inserted through an incision in the skin and maneuvered through the body to the deployment location. The stent is then expanded to an appropriately-sized expanded state so as to engage the inner walls of the treated vessel and to thus maintain patency thereof.

A first type (of stent is the self-expanding stent. When the stent is at the deployment location, the stent is released from the catheter and allowed to expand to an expanded state, in a manner analogous to that of a compressed spring. Self-expanding stents have been disclosed, for example, in U.S. Pat. Nos. 4,503,569; 4,580,568; 4,787,899; and 5,104,399.

A second type of stent is expanded from the unexpanded state to an expanded state using an expansion device, typically a catheter-borne balloon. When the stent is at the deployment location, the expansion device is activated inside the bore of the unexpanded stent to exert an outwards radial force on the inside of the stent, causing the stent to expand to a desired size. Such stents have been disclosed, for example, in U.S. Pat. Nos. 4,655,771; 4,733,665; 4,739,762; 4,800,882; 4,907,336; 4,994,071; 5,019,090; 5,035,706; 5,037,392; and 5,147,385.

As is known to one skilled in the art, many bodily vessels are bifurcated. By “bifurcated” is meant an object that splits to two branches along a length of the object. Two types of bifurcated objects (bifurcated blood vessels) having two different types of bifurcation are depicted in FIGS. 1A and 1B. A first type of bifurcated object 10 depicted in FIG. 1A includes a trunk vessel 12 from which a branch vessel 14 branches downstream from a bifurcation point 16. Generally, but not necessarily, the bore of branch vessel 14 is smaller than that of trunk vessel 12. A second type of bifurcated object 18 depicted in FIG. 1B includes a trunk vessel 12 from which two branch vessels 14A and 14B branch downstream from bifurcation point 16. Generally, but not necessarily, the bores of branch vessels 14A and 14B are smaller than the bore of trunk vessel 12. Branch vessel 14B of bifurcated object 18 is also bifurcated: branch vessel 14B is a trunk vessel from which branch vessel 20 branches. Although many naturally occuring vessels can be clearly classified as having either the first or the second type of bifurcation there are cases where such classification is ambiguous as there is no exact delineation of characteristics distinguishing one of the two types of bifurcation from the other.

In the field of medicine it is known to deploy stents in a bifurcated bodily vessel. Often, both branches of a bifurcated bodily vessel must be treated as damage, lesions or athersclerotic disease are often found in both the trunk and the branch vessels. However, even in cases where a branch vessel is healthy, interventional manipulation of the trunk vessel often compromises the integrity of the branch vessel. It is therefore preferable to deploy more than one stent in a bifurcated vessel, see for example U.S. Pat. Nos. 4,994,071; 5,669,924; 5,723,004; 5,906,640 or PCT patent application No. PCT/IB98/00496 published as WO 99/15103 of the inventor.

The challenges in deploying stents in a bifurcated vessel are generally a result of the fact that the component vessels of the bifurcated vessel are of different sizes and that the component vessels or the vicinity of the bifurcation point are often not flexible, structurally weak and susceptible to tearing due to damage, disease or age.

Ideally:

a) each individual stent is expanded independently to an appropriate extent to provide a bore of the desired size to the branch or trunk vessel in which deployed;
b) care is taken when expanding a stent in a larger branch or trunk vessel not to damage or cause catastrophic failure of a proximate smaller branch vessel or in the vicinity of the bifurcation point;
c) the stents are mechanically uncoupled so that movement, orientation and expansion of each individual stent is completely free and manipulation of one stent does not influence or effect another. This freedom of motion is preferably to a great extent as possible allowing the greatest possible of variation of divergence angle and relative orientation of one stent to another;
d) there is no pinching of bodily parts between any two stents;
e) the vessel is physically supported in the vicinity of the bifurcation point to prevent collapse of the vessel at the bifurcation point, but without excess force that may lead to rupture of the bifurcation point;
f) care is taken to ensure that flow through the treated vessels and the bifurcation point is unimpeded and not turbulent; and
g) once deployed the orientation between the two stents can change together with the natural movement of the organ (e.g., beating of the heart) in which deployed to prevent structural stress of the bifurcated vessel.

Strategies for treating a bifurcated vessel by deploying multiple independent stents in each branch or trunk vessel are summarized in U.S. Pat. No. 5,669,924. In a bifurcated blood vessel 10 as depicted in FIG. 1A, a branch-supporting stent is deployed in branch vessel 14 from C-C to the beginning of branch vessel 14 and subsequently a trunk-supporting stent is deployed in trunk vessel 12 both upstream and downstream of bifurcation point 16 from A-A to B-B. In a bifurcated blood vessel such as 18 as depicted in FIG. 1B, three individual stents are deployed, one branch-supporting stents in each of branch vessels 14A (from the bifurcation to B-B) and 14B (from the bifurcation to C-C) as well as a trunk-supporting stent in trunk vessel 12 (from the bifurcation to A-A).

Deployment of separate stents is problematic for a number of reasons. For support in the vicinity of the bifurcation point 16 it is necessary to deploy the stents close to bifurcation point 16, yet this gives no support to the immediate vicinity of bifurcation point 16. In some cases, one end of branch-supporting stent deployed in branch vessel 14 protrudes into trunk vessel 12, leading to turbulent flow through trunk vessel 12 and into branch vessel 14, (increasing the chances for restenosis) or making it impossible to deploy a trunk-supporting stent in trunk vessel 12. Further, if the stents are too close together, one stent may tangle with another or parts of the blood vessels may be pinched between the two stents. Expansion of multiple stents that are very close one to the other can lead to extreme stress and tearing of the blood vessels. To avoid such problems, the stents must be deployed far from each other, creating a large unsupported area in the vicinity of bifurcation point 16. Additionally, when treating vessels such as a bifurcated blood vessel 10 in FIG. 1A, a wall of a trunk-supporting stent deployed in trunk vessel 12 between A-A and B-B necessarily interferes with flow into branch vessel 14.

Another strategy discussed in U.S. Pat. No. 5,669,924 is the deployment of a stent assembly made up of two interleaved stents in blood vessels such as bifurcated blood vessel 18 depicted in FIG. 1B. In such a stent assembly, a downstream end of a first stent is deployed in branch vessel 14A, a downstream end of a second stent is deployed in branch vessel 14B and both upstream ends are interleaved and deployed in trunk vessel 12. Such a strategy is inadequate for a number of reasons. Since the ends of the stents deployed in branch vessels 14A and 14B offer less resistance to an expansion device than do the interleaved ends deployed in trunk vessel 12, the parts of stents in branch vessels 14A and 14B are expanded to a greater extent than the parts of the stents deployed in trunk vessel 12, even though trunk vessel 12 generally has a larger bore than do branch vessels 14A and 14B. Further, only certain types of stents are interleavable, limiting the applicability of such a strategy. Further, the interleaving of the two stents generally interferes with the proper expansion of the stents, leading to suboptimal support of vessel 18 and turbulent flow therethrough. Further, parts of vessel 18 may be pinched between the stents.

A number of bifurcated stent assemblies, generally comprising two or more conjoined stents are known in the art. Herein the freedom of independent motion of the conjoined stents, is termed bendability. The term in-plane bendability refers to independence of motion of any two stents within the plane defined by the axes of the two stents, especially with regards to the angle of divergence of the stents. The term out-of-plane bendability refers to independence of motion of any two stents outside of the plane defined by the axes of the two stents. A stent assembly having a high degree of bendability is one where the motion of a first stent of a stent assembly relative to a second stent of the assembly is possible to a high degree without bending, distorting or buckling of the second stent.

In U.S. Pat. No. 4,994,071 is taught a bifurcated stent assembly substantially comprising a single trunk-supporting stent having two axial backbone wires with two branch-supporting stents connected to the trunk-supporting stent, each branch-supporting stent to one of the two axial backbone wires. The stent assembly of U.S. Pat. No. 4,994,071 is suitable for deployment in vessels similar to vessel 18 depicted in FIG. 1B but no embodiment suitable for deployment in vessel 10 depicted in FIG. 1A is disclosed. A disadvantage of the stent assembly of U.S. Pat. No. 4,994,071 is that little support is given to the vicinity of a bifurcation point 16. A further disadvantage is that the structure of the stent assembly allows for pinching of parts of the bodily vessel between the two branch-supporting stents. A further disadvantage is that the bendability of the stent assembly is low as the component stents are mechanically coupled through the axial backbone wires. A further disadvantage is that the bendability of the stent assembly is low as the bendability arises from the flexibility of the axial backbone wires which is homogenous along the length of a given wire. As a result, a bending force applied to one stent flexes that stent rather than changing the relative orientation of two component stents.

In order to overcome some of the disadvantages of the bifurcated expandable stent assembly of U.S. Pat. No. 4,994,071, U.S. Pat. No. 5,723,004 teaches a bifurcated expandable graft secured to the bifurcated stent assembly of U.S. Pat. No. 4,994,071. The graft is made up of a flexible liner and/or a flexible jacket applied to the stent assembly. The graft prevents pinching between the component stents and provides a matrix for cell growth so that ultimately the vicinity of the bifurcation growth is mechanically strengthened by the new cells. The graft allegedly provides support to the vicinity of the bifurcation point. That said, the liner/jacket does not have a structural role in the stent assembly which is provided exclusively by the stents. As a result, the component stents must be associated through axial backbone wires as described above so the limited bendability and the mechanical coupling of the stents remain. Further, in U.S. Pat. No. 5,723,004 the expensive and complex methods disclosed for producing the stent assembly limit the utility of the teachings: a liner is applied to a mandrel (e.g., by electrospinning, dipping or fiber winding of synthetic materials), a stent is crimped over the liner on the mandrel and subsequently a jacket is applied over the stent held on the mandrel. Further, the teachings of U.S. Pat. No. 5,723,004 are directed exclusively to permeable synthetic grafts to allow cell growth therethrough. No solid, non-permeable graft is disclosed nor is any use of non-synthetic materials suggested. Further, the thickness of the graft is not discussed but, as is seen in the figures is quite significant: such a thick graft either leads to the stent assembly having a small bore and thus forming a flow bottleneck once deployed, or requires over-expansion which may cause the vessel in which the stent assembly is deployed to rupture.

In PCT patent application No. PCT/IB98/00496 published as WO 99/15103 of the inventor is taught an articulated stent assembly. To the side of a trunk stent is attached a branch stent by some method or mechanism that substantially defines an articulation or joint, e.g., a hinge. For deployment, the divergence angle of the branch stent relative to the trunk stent is varied as desired at the joint with substantially no distortion or buckling of either stent. The close coupling of the component stents ensures that support is given to the bifurcation point. A disadvantage of the teachings of PCT patent application No. PCT/IB98/00496 is that not all stents may be suitable to be coupled using an articulation or joint.

It would be highly advantageous to have a bifurcated stent assembly useful for deployment in bifurcated vessels and not having at least some of the disadvantages of the prior art.

### SUMMARY OF THE INVENTION

The present invention successfully addresses at least some of the shortcomings of the prior art by providing a method for bendably associating two stents and a bifurcated stent assembly for deployment in a bifurcated bodily vessel.

The present invention is based in the use of a flexible membrane, preferably a tubular membrane, as a stent connector to bendably hold and functionally associate two stents that are components of a bifurcated stent assembly. The flexible stent connector of the present invention allows the relative orientation of the two stents to be varied with little if any deformation of the connected stents. In preferred embodiments, the stent connector is configured to provide support to the bodily vessel in the vicinity of the bifurcation and to prevent pinching of bodily parts between the two stents.

According to the teachings of the present invention there is provided a method for bendably associating a first expandable stent with a second expandable stent for deployment in a bifurcated vessel comprising using a flexible membrane as a stent connector to connect the second expandable stent to the first expandable stent so that the bore of the first expandable stent defines an elongated trunk volume and the bore of the second expandable stent defines an elongated branch volume branching from the trunk volume wherein the two stents are substantially devoid of direct physical association. In an embodiment of the present invention the two stents are also devoid of mutual physical contact.

In an embodiment of the present invention, the stent connector comprises at least one filament. By filament is meant that at least part of the stent connector has an elongated shape such as that of a band, filament, ribbon, strand, string, strip or thread.

In a preferred embodiment of the present invention, at least part of the stent connector is substantially tubular.

In an embodiment of the present invention, the stent connector includes a substantially tubular trunk part functionally associated with the first expandable stent. In an embodiment of the present invention the substantially tubular trunk part of the stent connector surrounds at least part of the first expandable stent. In an embodiment of the present invention the substantially tubular trunk part of the stent connector is disposed within the bore of the first expandable stent.

In an embodiment of the present invention, the stent connector includes a substantially tubular branch part functionally associated with the second expandable stent. In an embodiment of the present invention the substantially tubular branch part of the stent connector surrounds at least part of the second expandable stent. In an embodiment of the present invention at least part of the substantially tubular branch part of the stent connector is disposed within the bore of the second expandable stent.

In an embodiment of the present invention the stent connector includes a first substantially tubular trunk part functionally associated with the first expandable stent and a second substantially tubular branch part functionally associated with the second expandable stent, where the branch part of the stent connector branches from the trunk part of the stent connector.

In an embodiment of the present invention the branch volume is in fluid communication with the trunk volume from a side of the trunk volume, preferably through an opening in the side of the first expandable stent.

In an embodiment of the present invention the branch volume is in fluid communication with the trunk volume from an end of the trunk volume.

According to the teachings of the present invention there is also provided a bifurcated stent assembly, comprising a) a flexible membrane as a stent connector; b) a first expandable stent functionally associated with the stent connector, wherein the bore of the first expandable stent defines an elongated trunk volume; and c) a second expandable stent functionally associated with the stent connector, wherein the bore of the second expandable stent defines an elongated branch volume branching from the trunk volume wherein the stents are substantially devoid of direct physical association. In an embodiment of the present invention the two stents are also devoid of mutual physical contact. In an embodiment of the present invention, the stent connector comprises at least one filament.

In an embodiment of the present invention the branch volume is in fluid communication with the trunk volume from a side of the trunk volume. In an embodiment of the present invention, the first expandable stent is provided with a side opening through which the branch volume is in fluid communication with the trunk volume.

In an embodiment of the present invention the branch volume is in fluid communication with the trunk volume from an end of the trunk volume.

In an embodiment of the present invention one, some or all of the component stents of a bifurcated stent assembly of the present invention is secured to the stent connector by at least one member of the group consisting of sutures, adhesives, bending members, clamps, glue, hooks, piercing members and staples.

In a preferred embodiment of the present invention at least part of the stent connector of a bifurcated stent assembly of the present invention is substantially tubular.

In an embodiment of the. present invention, the stent connector includes a substantially tubular trunk part functionally associated with the first expandable stent.

In an embodiment of the present invention, the substantially tubular trunk part of the stent connector surrounds at least part of the first expandable stent. In an embodiment of the present invention, the first expandable stent is substantially entirely contained within the stent connector, especially a tubular part thereof, especially the trunk part thereof. In an embodiment of the present invention, at least one and preferably both of the ends of the first expandable stent emerge from the stent connector.

In an embodiment of the present invention, at least part of the trunk part of the stent connector is disposed within the bore of the first expandable stent. In embodiments of the present invention, the first expandable stent is entirely outside the stent connector. In an embodiment of the present invention, the trunk part of the stent connector is substantially entirely disposed within the bore of the first expandable stent. In an embodiment of the present invention, either one or both ends of the substantially tubular trunk part of the stent connector emerge from an end of the first expandable stent, and preferably are folded over a respective end.

In an embodiment of the present invention, the stent connector includes a substantially tubular branch part functionally associated with the second expandable stent.

In an embodiment of the present invention, the branch part of the stent connector surrounds an end of the second expandable stent.

In an embodiment of the present invention, the second expandable stent is substantially entirely contained within the stent connector, especially a tubular part thereof, especially the branch part thereof. In an embodiment of the present invention, an end of the second expandable stent emerges from the stent connector.

In an embodiment of the present invention, at least part of the branch part of the stent connector is disposed within the bore of the second expandable stent. In an embodiment of the present invention, the second expandable stent is entirely outside the stent connector. In an embodiment of the present invention, the branch part of the stent connector is substantially entirely disposed within the bore of the second expandable stent. In an embodiment of the present invention, an end of the substantially tubular branch part of the stent connector emerges from an end of the second expandable stent, and is preferably folded over the respective end.

In a preferred embodiment of the present invention the stent connector includes at least two substantially tubular parts: a first substantially tubular trunk part functionally associated with the first expandable stent and a second substantially tubular branch part functionally associated with the second expandable stent, where the branch part branches from the trunk part. In a preferred embodiment of the present invention, the first expandable stent is provided with a side opening, the stent connector is at least partially disposed within the bore of the first expandable stent so that the branch part of the stent connector emerges from the side opening of the first expandable stent.

In embodiments of the present invention, a bifurcated stent assembly of the present invention is made up of more than two expandable stents. Generally, additional stents are all stents that, analogously to the second expandable stent, branch from the first expandable stent.

In an embodiment of the present invention, a bifurcated stent assembly of the present invention further comprises a third expandable stent functionally associated with the stent connector, wherein the bore of the third expandable stent defines an elongated second branch volume branching from the trunk volume. In an embodiment of the present invention, the second branch volume is in fluid communication with the trunk volume from a side of the trunk volume, preferably through an opening in the side of the first expandable stent. In an embodiment of the present invention the second branch volume is in fluid communication with the trunk volume from an end of the trunk volume. In an embodiment of the present invention, the stent connector includes a substantially tubular branch part functionally associated with the third expandable stent.

In an embodiment of the present invention, the stent connector is substantially a vessel comprising walls of a flexible material and at least three ports, a first port, a second port and a third port, and, wherein the first expandable stent is functionally associated with the first port and the second expandable stent is functionally associated with the third port. In an embodiment of the present invention, the first expandable stent is functionally associated with both the first port and the second port. In an embodiment of the present invention, the bifurcated stent assembly further comprises a third expandable stent functionally associated with the second port. In an embodiment of the present invention, the stent connector has exactly three ports.

In an embodiment of the present invention, the first port, the second port and the walls of the stent connector therebetween define a trunk part of the stent connector that encloses a volume that substantially overlaps the trunk volume of the stent assembly.

In an embodiment of the present invention, the first expandable stent is provided with a side opening. In an embodiment of the present invention, the side opening of the first expandable stent is functionally associated with the third port of the stent connector. In an embodiment of the present invention, the bore of the second expandable stent is in fluid communication with the bore of the first expandable stent through the side opening.

In an embodiment of the present invention, the first port, the second port, the walls of the stent connector therebetween together with the first expandable stent substantially define at least part of a trunk implant, that is the part of the bifurcated stent assembly that when used is deployed in the trunk vessel.

In an embodiment of the present invention, the third port is in fluid communication with the trunk volume through a substantially tubular branch of the stent connector. In an embodiment of the present invention, the third port, the walls of the substantially tubular branch of the stent connector together with the second expandable stent substantially define at least part of a branch implant, that is the part of the bifurcated stent assembly that when used is deployed in the branch vessel.

In an embodiment of the present invention, at least part of the tubular branch is contained within the bore of the second expandable stent. In an embodiment of the present invention, the tubular branch is substantially entirely contained within the bore of the second expandable stent. In an embodiment of the present invention, a part of the tubular branch emerges from an end of the second expandable stent and is preferably folded thereover.

In an embodiment of the present invention, the flexible membrane of the stent connector is substantially elastic.

In an embodiment of the present invention, the flexible membrane of the stent connector is substantially impermeable to fluids.

In an embodiment of the present invention, the flexible membrane of the stent connector is substantially impervious to tissue proliferation therethrough. Such imperviousness is useful in preventing tissue buildup on and through the membrane and prevents the migration of smooth muscle cells. Such an embodiment is useful for providing a treated bodily vessel with a new, undamaged, smooth lining that is substantially impervious to restenosis.

In an embodiment of the present invention, the flexible membrane of the stent connector is permeable. Such an embodiment is useful as the membrane permeability allows cells to grow into and through the membrane, making the membrane substantially part of the vessel in which deployed.

Generally, the walls of a stent connector of the present invention are as thin as possible to ensure that a respective bifurcated stent assembly be flexible, have a low profile during navigation to the deployment site and to restrict a vessel in which deployed as little as possible and at the same time the walls must be sufficiently elastic and strong to permit navigation and deployment without tearing. Clearly, the nature of the material from which a given stent connector is made determines in part the thickness of the walls. That said, a stent connector preferably has walls that are not thicker than about 0.75 mm, not thicker than about 0.45 mm, not thicker than about 0.25 mm, not thicker than about 0.20 mm, and even not thicker than about 0.05 mm.

In an embodiment of the present invention, the stent connector and especially the stent connector wall are substantially fashioned from a synthetic or polymeric material including but not limited to polytetrafluoroethylene, urethane, elastomer, polyamide (e.g., Nylon) and polyester (e.g., Dacron).

In an embodiment of the present invention, the stent connector and especially the stent connector wall are substantially fashioned from biological tissue including but not limited to autologous tissue, heterologous tissue, venous tissue, arterial tissue, serous tissue, pleura, peritoneum, pericardium and aortic leaflet. In a preferred embodiment, the flexible membrane is a section of bifurcated biological tissue such as a bifurcated blood vessel (including a bifurcation) that is harvested and treated so as to prepare a seamless one-piece stent connector. In an embodiment of the present invention, the material is harvested from a source selected from the group consisting of human sources and non-human animal sources, especially equine, porcine, bovine and human. In an embodiment of the present invention the material is thinned, where after harvesting one or more layers of the harvested tissue is removed, e.g., by scraping, shaving, slicing or skiving. In an embodiment of the present invention, the material (or components of the material such as collagen) is cross-linked, for example by treatment with a glutaraldehyde or a phosphate solution.

In an embodiment of the present invention, the stent connector is substantially fashioned from serous membrane including a serous tissue stratum and a basement tissue stratum, where the serous membrane is, for example, from porcine, bovine, equine or human serous membrane.

In a preferred embodiment of the present invention, the stent connector is substantially fashioned from a thinned serous membrane, where a harvested serous membrane (peritoneum, pericardium or pleural tissue especially porcine, bovine, equine and human serous tissue) has been processed by removal of a layer of at least some of the associated basement tissue (and thus thinned), preferably removal of all the basement tissue, leaving only the serous tissue layer. Thus in an embodiment of the present invention, the stent connector comprises a thinned serous membrane including a serous tissue stratum and a basement tissue stratum, wherein the thinned serous membrane has been processed by removal of a layer of basement tissue from a harvested serous membrane. In an embodiment of the present invention only a layer of the basement tissue is removed. In an embodiment of the present invention, the thinned serous membrane is substantially serous tissue devoid of basement tissue. In an embodiment of the present invention, the material consists essentially of serous tissue.

When serous tissue is used to implement the teachings of the present invention it is usually preferred to orient the membrane so that the smooth serous strata is facing the fluid flow to reduce turbulent flow.

In embodiments of the present invention, one or more of the component stents of the bifurcated stent assembly of the present invention are jacketed, for example with any of the jackets known in the art.

In embodiments of the present invention, one or more of the component stents of the bifurcated stent assembly of the present invention are coated, for example with any of the stent coatings known in the art.

Two parameters used when selecting a stent for use in implementing the teachings of the present invention are the expanded and unexpanded diameters of the stent.

Generally it is important that the unexpanded diameter of a stent be as small as possible to ease navigation through the body to the deployment location. That said, the unexpanded diameter must be large enough to allow threading of the stent onto a deployment catheter and, for not-self expanding stents, a stent-expanding device such as a stent-expanding balloon. Although there may be some variation in the unexpanded diameter of even two identical stents depending on how the two stents are used, herein by unexpanded diameter is intended the outer diameter of an expandable stent when crimped to smallest practical diameter onto a delivery catheter for deployment.

In embodiments of the present invention, a first expandable stent, a second expandable stent (and third or more expandable stents if present) are of substantially similar or identical dimensions, especially length, expanded diameter and/or unexpanded diameter.

In embodiments of the present invention a first expandable stent, a second expandable stent (and third or more expandable stents if present) are of substantially different dimensions, especially length, expanded diameter and/or unexpanded diameter. In cases where the first expandable stent, the second expandable stent (and third or more expandable stents if present) are of different dimensions, generally the first expandable stent is larger (especially of larger expanded and/or unexpanded diameter) as it is generally the first expandable stent that is destined to be deployed in a trunk vessel rather than in a smaller-bored branch vessel.

In embodiments of the present invention, the diameter (expanded or unexpanded) of the first expandable stent is substantially similar to the respective diameter of the second expandable stent (and third or more expandable stents if present).

In embodiments of the present invention, the diameter (expanded or unexpanded) of the first expandable stent is larger than the respective diameter of the second expandable stent (and third or more expandable stents if present).

Generally, any given stent has a wide range of expanded diameters larger than a respective unexpanded diameter. The expanded diameter of a stent subsequent to deployment is determined by the user of the stent according to medical criteria including the natural size of the lumen of the vessel in which the stent is deployed. That said, self-expanding stents are characterized by a specific maximal expansion that is the maximal diameter of the stent when the stent is free from externally applied forces. Most non-self expanding stents are also characterized by a maximal expansion that is the greatest extent to which the stent is expandable without comprising the structural integrity thereof.

Generally, a first expandable stent and a second expandable stent of a bifurcated stent assembly of the present invention are relatively close together. Thus, in embodiments of the present invention the distance between the first expandable stent and the second expandable stent is no greater than about four, no greater than about three, no greater than about two, no greater than about one and even no greater than about half of an unexpanded diameter of the second expandable stent.

According to the teachings of the present invention there is also provided a method of preparing a bifurcated stent assembly of the present invention, generally comprising providing an appropriate stent connector and a required number of stents and assembling the components in accordance with the description and the figures herein. In embodiments of the present invention, tubular components of a stent connector are made of substantially tubular tissue harvested as is.

In embodiments of the present invention, a stent connector comprises conjoined tubular components. In embodiments of the present invention, one or more tubular components are seamless, for example of harvested tubular tissue. In embodiments of the present invention, bifurcated tubular components of a stent connector are made of harvested substantially bifurcated tubular tissue harvested. In embodiments of the present invention, tubular components of a stent connector are made of substantially planar sheet of material that is fashioned into a tube, for example by overlapping or abutting two edges of the sheet. In embodiments of the present invention, a tubular wall of a stent connector is made of a first material while other components are made of a second material.

In an embodiment of the present invention, a stent connector of the present invention is essentially fashioned from one sheet, preferably planar or substantially planar, of an appropriate membrane (synthetic or harvested) rolled up into shape (with abutting or overlapping edges) and then fixed in shape using any of the methods known in the art. Preferred shapes of sheets suitable for use in fashioning a stent connector from one sheet of material generally include shapes having at least one C2 symmetry axis and/or at least one external angle of no greater than 90° and/or at least 7 sides. Suitable shapes include cross shapes, “T”-shapes, “W”-shapes, “X”-shapes, “Y”-shapes and “Ψ”-shapes.

According to the teachings of the present invention there is also provided a method of treatment, for example of an aneurism, generally comprising deploying a bifurcated stent assembly of the present invention inside the lumen of a bifurcated bodily vessel, especially in the cardiovascular, cerebrovascular or peripheral vascular system. Such methods of treatment include relining a vessel, supporting patency of a vessel, treating an aneurism or avoiding bursting of a vessel.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B are depictions of bifurcated objects in which deployment of a bifurcated stent assembly of the present invention is useful;

FIGS. 2A and 2B are depictions of bifurcated stent assemblies of the present invention including strip-like stent connectors;

FIGS. 3A and 3B are depictions of bifurcated stent assemblies of the present invention including stent connectors comprising a substantially tubular trunk part associated with a first expandable stent and a strip-like part associated with a second expandable stent;

FIGS. 4A and 4B are depictions of bifurcated stent assemblies of the present invention including stent connectors comprising a substantially tubular branch part associated with a second expandable stent and a strip-like part associated with a first expandable stent;

FIGS. 5A, 5B, 5C and 5D are depictions of bifurcated stent assemblies of the present invention including stent connectors comprising a substantially tubular trunk part associated with a first expandable stent and a substantially tubular branch part associated with a second expandable stent;

FIGS. 6A, 6B and 6C are depictions of bifurcated stent assemblies of the present invention including three expandable stents; and

FIGS. 7A-7G depict sheets of various shapes rolled-up and edges attached to fashion stent connectors of the present invention.

### DESCRIPTION OF EMBODIMENTS OF THE INVENTION

The present invention is of a bifurcated stent assembly useful for deployment in bifurcated bodily vessels. The present invention is also of a method for bendably associating two stents. The present invention is also if a method of treatment.

The principles, uses and implementations of the teachings of the present invention may be better understood with reference to the accompanying description and figures. Upon perusal of the description and figures present herein, one skilled in the art is able to implement the teachings of the present invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of medicine, biology, chemistry, material sciences and engineering. Such techniques are thoroughly explained in the literature. Unless otherwise defined, all technical and scientific terms used herein have the same meaning as commonly understood by one of ordinary skill in the art to which the invention belongs. In addition, the descriptions, materials, methods and examples are illustrative only and not intended to be limiting. Methods and materials similar or equivalent to those described herein can be used in the practice or testing of the present invention. All publications, patent applications, patents and other references mentioned are incorporated by reference in their entirety as if fully set forth herein. In case of conflict, the specification herein will control.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the relevant arts. Implementation of the methods of the present invention involves performing or completing selected tasks or steps manually, automatically, or a combination thereof.

The present invention is related to a bifurcated stent assembly useful for deployment in bifurcated bodily vessels including a trunk vessel and a branch vessel. The teachings of the present invention are based on the use of a flexible membrane, preferably a tubular membrane, as a stent connector to bendably hold and functionally associate two or more stents making up a bifurcated stent assembly of the present invention. Such a flexible stent connector allows two stents to be bendably associated so that the distance between the two stents is relatively fixed and the respective lumina are in unobstructed fluid communication but that the relative orientation of the axes of the stents is variable in virtually all directions with little deformation of the thus-associated stents.

In a preferred embodiment, the flexible membrane includes tubular sections that are associated with the substantially tubular component stents, provide support or relining of the vessel in which deployed, reduce turbulent flow in the vessel subsequent to deployment and, depending on the configuration, avoid or prevent pinching of the vessel interior during deployment.

Specifically, the teachings of the present invention relate to an innovative method of associating two expandable stents by using a flexible membrane as a stent connector. A stent assembly of the present invention comprises a stent connector functionally associated with a first expandable stent the bore of which defines an elongated trunk volume and a second expandable stent the bore of which defines an elongated branch volume, where the branch volume branches from the trunk volume.

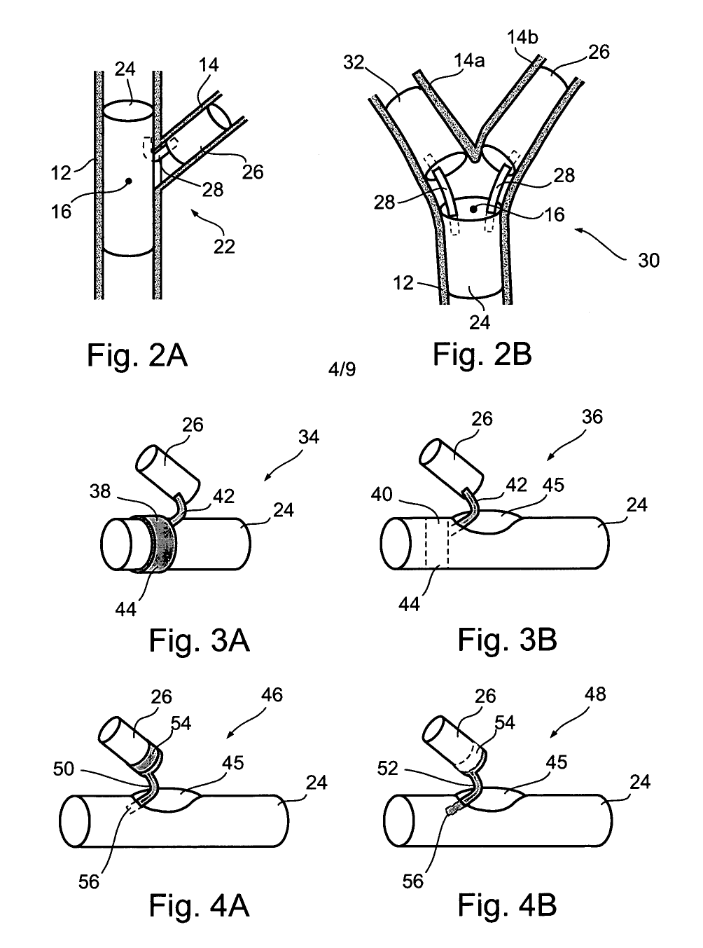

In FIGS. 2A and 2B are depicted two bifurcated stent assemblies of the present invention where expandable stents are associated in accordance with the teachings of the present invention.

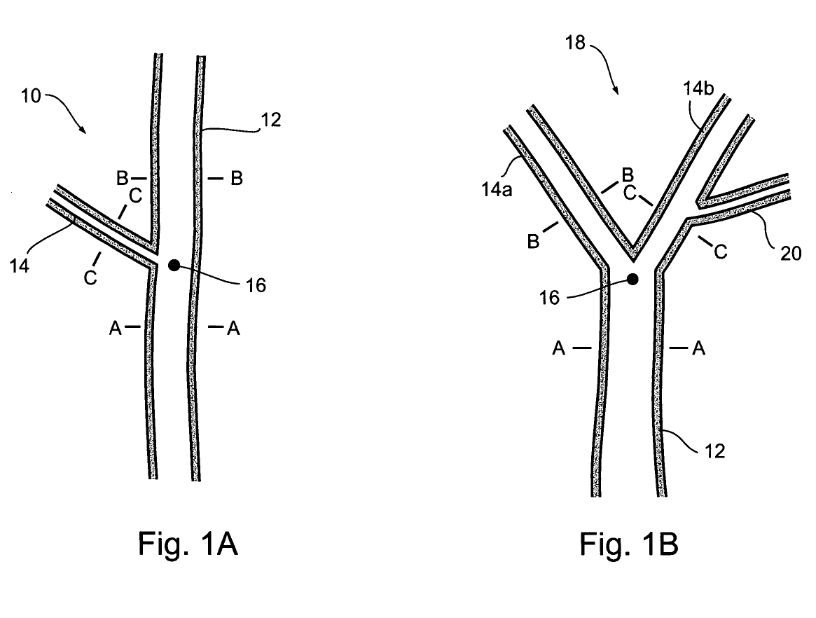

In FIG. 2A, a bifurcated stent assembly 22 is made up of a first expandable stent 24, a second expandable stent 26 and a stent connector 28 which is substantially a strip of heterologous tissue (e.g., serous tissue, such as from thinned equine pericardium). Stent assembly 22 may be deployed in a bifurcated artery such as 10 depicted in FIG. 1A made up of a trunk artery 12 and a branch artery 14. Stent connector 28 is secured to first expandable stent 24 and second expandable stent 26 by sutures (or optionally by sutures, adhesives, bending members, clamps, glue, hooks, piercing members, staples, other applicable mechanical mean or combinations thereof).

First expandable stent 24 is deployed in and supports patency of trunk artery 12 both upstream and downstream of bifurcation point 16 while second expandable stent 26 is deployed in and supports patency of branch artery 14. The bore of first expandable stent 24 defines an elongated trunk volume and the bore of second expandable stent 26 defines an elongated branch volume branching from the trunk volume. As is seen in FIG. 2A, the branch volume is in fluid communication with the trunk volume from the side of first expandable stent 24.

Since first expandable stent 24 and second expandable stent 26 are devoid of mutual physical association or contact and attached only through stent connector 28, manipulation and expansion of either stent during deployment of stent assembly 22 does not cause deformation such as buckling or stretching of the other stent.

In FIG. 2B, a bifurcated stent assembly 30 is made up of a first expandable stent 24, a second expandable stent 26, a third expandable stent 32 and two stent connectors 28 which are substantially strips of heterologus tissue (e.g., serous tissue such as from thinned porcine pericardium). Stent assembly 30 is deployed in a bifurcated artery such as 18 depicted in FIG. 1B made up of a trunk artery 12 that branches to two substantially similar sized branch arteries 14A and 14B. Stent connectors 28 are secured to first expandable stent 24, second expandable stent 26 and third expandable stent 32 by sutures (or optionally by adhesives, bending members, clamps, glue, hooks, piercing members, staples, other applicable mechanical mean or combinations thereof). First expandable stent 24 is deployed in and supports patency of trunk artery 12 upstream of bifurcation point 16, second expandable stent 26 is deployed in and supports patency of branch artery 14A and third expandable stent 23 is deployed in and supports patency of branch artery 14B. The bore of first expandable stent 24 defines an elongated trunk volume and the bores of second expandable stent 26 and third expandable stent 32 each define an elongated branch volume branching from the trunk volume. As is seen in FIG. 2B the two branch volumes are in fluid communication with the trunk volume from an end of first expandable stent 24.

Since first expandable stent 24, second expandable stent 26 and third expandable stent 32 are devoid of mutual physical association or contact and attached only through stent connectors 26, manipulation and expansion of any one stent during deployment of stent assembly 30 does not cause any deformation such as buckling or stretching in the other stents.

In FIGS. 2A and 2B, stent connectors 26 of bifurcated stent assemblies 22 and 30 are strips of heterologous tissue (e.g., serous tissue, pleura, peritoneum, pericardium), but in other non-depicted embodiments are also bands, filaments, ribbons, strands, strings, strips or threads of any other suitable material.

In preferred embodiments of the present invention, at least part of a stent connector of a bifurcated stent assembly of the present invention is substantially tubular (including ring-shaped) allowing better association with one, two or more of the component tubular stents making up a given stent assembly. Better association includes such aspects as increased contact area between a stent and the respective tubular part of the stent connector reducing the chance of stent connector tearing, distribution of potentially deforming forces over a large area of a stent, better securing of the stent connector to an individual stent and more accurate relative positioning of any two stents.

The component stents of a stent assembly of the present invention are preferably associated with a tubular part of a stent connector. As is discussed in detail hereinbelow, embodiments of stent assemblies of the present invention made up of two stents have a stent connector with only one tubular part that one tubular part associated with a first stent or have a stent connector with only one tubular part that tubular part associated with a second stent. A preferred embodiment of a stent assembly of the present invention has a stent connector with two tubular parts, a first tubular part associated with a first stent and a second tubular part associated with a second stent. Embodiments of stent assemblies of the present invention made up of three or more stents preferably have a stent connector comprising a tubular part associated with each one of the component stents.

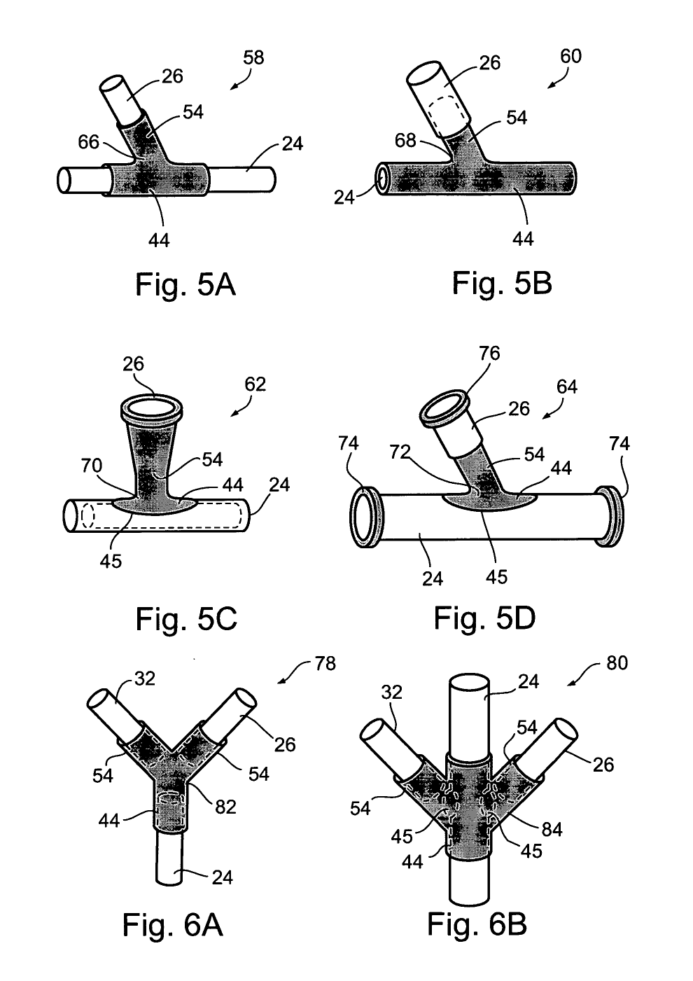

In FIGS. 3A and 3B are depicted stent assemblies of the present invention, 34 and 36 respectively including stent connectors 38 and 40 respectively. Stent connectors 38 and 40 each have a strip-like branch part 42 associated with a second expandable stent 26 secured thereto by sutures (or optionally by adhesives, bending members, clamps, glue, hooks, piercing members, staples, other applicable mechanical mean or combinations thereof) analogously to stent connectors 28 of stent assemblies 22 and 30. Stent connectors 38 and 40 each have a substantially tubular trunk part 44 (depicted in phantom in FIG. 3B) associated with a first expandable stent 24. In such a way, second expandable stent 26 is associated with first expandable stent 24 at a desired distance but with virtually no other limitations to the relative orientations of the two stents. Stent connectors 38 and 42 are made of a thinned layer of heterologous tissue preferably bovine pericardium (preferably thinned so as to consist essentially of serous tissue, see U.S. Pat. Nos. 6,468,300 and 6,254,627 of the inventor) known for strength, tear-resistance, biocompatibility, expandability and elasticity. In both cases first expandable stent 24 is destined for deployment in a trunk artery while second expandable stent 26 is destined for deployment in a branch artery.

In FIG. 3A, tubular trunk part 44 of stent connector 38 is disposed entirely on the outside of and surrounding first expandable stent 24, and is held in place by tension and the attendant friction. Tubular trunk part 44 expands together with first expandable stent 24 during expansion thereof. Strip-like branch part 42 of stent connector 38 is disposed entirely on the inside of the bore of second expandable stent 26.

In FIG. 3B, first expandable stent 24 is provided with a side opening 45. Tubular trunk part 44 of stent connector 40 is disposed entirely on the inside the bore of and surrounded by first expandable stent 24, and is held in place by sutures (or optionally by adhesives, bending members, clamps, glue, hooks, piercing members, staples, other applicable mechanical mean or combinations thereof). Tubular trunk part 44 expands together with first expandable stent 24 during expansion thereof. Strip-like branch part 42 of stent connector 40 emerges from side opening 45 and is disposed entirely on the outside of second expandable stent 26.

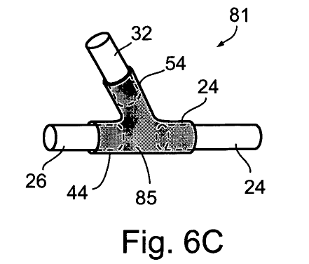

In FIGS. 4A and 4B are depicted stent assemblies of the present invention, 46 and 48 respectively including stent connectors 50 and 52 respectively. Stent connectors 50 and 52 each have a strip-like trunk part 56 associated with a first expandable stent 24 and secured thereto by sutures (or optionally by adhesives, bending members, clamps, glue, hooks, piercing members, staples, other applicable mechanical mean or combinations thereof) analogously to stent connectors 28 of stent assemblies 22 and 30. Stent connectors 50 and 52 each have a substantially tubular branch part 54 (depicted in phantom in FIG. 4B) associated with a second expandable stent 26. In such a way, second expandable stent 26 is associated with first expandable stent 24 at a desired distance but with virtually no other limitations to the relative orientations of the two stents. Stent connectors 50 and 52 are made of a thinned layer of heterologous tissue preferably bovine pericardium (preferably thinned so as to consist essentially of serous tissue, see U.S. Pat. Nos. 6,468,300 and 6,254,627 of the inventor) known for strength, tear-resistance, biocompatibility, expandability and elasticity. In both cases first expandable stent 24 is destined for deployment in a trunk artery while second expandable stent 26 is destined for deployment in a branch artery.

In FIG. 4A, first expandable stent 24 is provided with a side opening 45. Tubular branch part 54 of stent connector 50 is disposed entirely on the outside of and surrounding second expandable stent 26, and is held in place by tension and the attendant friction. Tubular branch part 54 expands together with second expandable stent 26 during expansion thereof. Strip-like trunk part 56 of stent connector 50 passes through side opening 45 of first expandable stent 24 and is disposed entirely inside the bore of first expandable stent 24.

In FIG. 4B, first expandable stent 24 is provided with a side opening 45. Tubular branch part 54 of stent connector 36 is disposed entirely inside the bore of and is substantially contained within second expandable stent 26, and is held in place by sutures (or optionally by adhesives, bending members, clamps, glue, hooks, piercing members, staples, other applicable mechanical mean or combinations thereof). Tubular branch part 54 expands together with second expandable stent 24 during expansion thereof. Strip-like trunk part 56 of stent connector 52 is disposed entirely on the outside of first expandable stent 24.

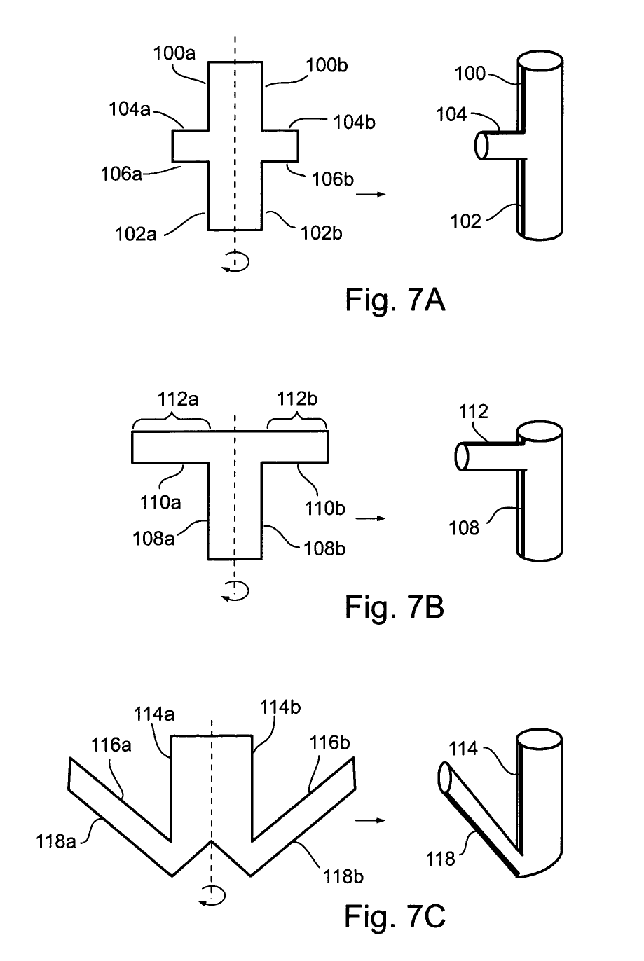

In FIGS. 5A, 5B, 5C and 5D are depicted stent assemblies of the present invention, 58, 60, 62 and 64 respectively including stent connectors 66, 68, 70 and 72 respectively. Stent connectors 66, 68, 70 and 72 each have two substantially tubular parts, a tubular trunk part 44 associated with a first expandable stent 24 and a tubular branch part 54 branching from trunk part 44 and associated with a second expandable stent 26. In such a way, second expandable stent 26 is associated with first expandable stent 24 at a desired distance but with virtually no other limitations to the relative orientations of the two stents. When a tubular part 44 or 54 is disposed inside the bore of a respective stent, the tubular part is secured thereto by sutures (or optionally by adhesives, bending members, clamps, glue, hooks, piercing members, staples, other applicable mechanical mean or combinations thereof). When a tubular part 44 or 54 is disposed on the outside of a respective stent, the tubular part is preferably held in place by tension and the attendant friction (although in embodiments of the present invention the tubular part is held in place by sutures, adhesives, bending members, clamps, glue, hooks, piercing members, staples, other applicable mechanical mean or combinations thereof). A given tubular part expands together with a respective stent during expansion thereof.

In FIG. 5A, tubular trunk part 44 of stent connector 66 is on the outside of and surrounds part of first expandable stent 24. The two ends of first expandable stent 24 emerge from tubular trunk part 44. Tubular branch part 54 of stent connector 66 is on the outside of and surrounds part (an end) of second expandable stent 26. An end of second expandable stent 26 emerges from tubular branch part 54 of stent connector 66.

In FIG. 5B, tubular trunk part 44 of stent connector 68 is on the outside of and surrounds first expandable stent 24. First expandable stent 24 is substantially entirely contained within tubular trunk part 44 of stent connector 68. Tubular branch part 54 of stent connector 68 is disposed inside the bore of and is substantially entirely contained within second expandable stent 26.

In FIG. 5C, tubular trunk part 44 of stent connector 70 is disposed entirely inside the bore of first expandable stent 24. First expandable stent 24 is substantially entirely outside of tubular trunk part 44 of stent connector 68. Tubular branch part 54 of stent connector 70 emerges from side opening 45 of first expandable stent 24. Tubular branch part 54 of stent connector 70 entirely covers second expandable stent 26. Second expandable stent 26 is substantially entirely contained within tubular branch part 54 of stent connector 70.

In FIG. 5D, tubular trunk part 44 of stent connector 72 is disposed inside the bore of first expandable stent 24. The two ends of tubular trunk part 44 of stent connector 72 emerge from the ends of first expandable stent 24 and are folded over the respective end. Tubular branch part 54 of stent connector 72 emerges from side opening 45 of first expandable stent 24. Tubular branch part 54 of stent connector 72 is disposed inside the bore of second expandable stent 26. An end of tubular branch part 54 emerges from an end of second expandable stent 26 and is folded thereover. The folding allows for a simple to manufacture stent assembly of the present invention where stent connector 72 is securely attached to first expandable stent 24 and second expandable stent 26.

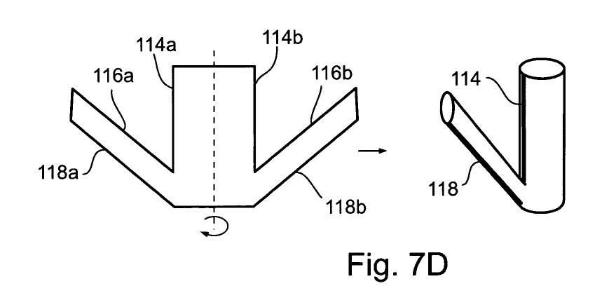

In FIG. 6A is depicted a stent assembly 78 of the present invention comprising stent connector 82. In FIG. 6B is depicted a stent assembly 80 of the present invention comprising stent connector 84. Stent assemblies 78 and 80 include three expandable stents, a first expandable stent 24 that is destined for deployment in a trunk vessel and a second expandable stent 26 and a third expandable stent 32 that are both destined for deployment in branch vessels. Stent connectors 82 and 84 include three substantially tubular parts, a tubular trunk part 44 associated with first expandable stent 24, and two tubular branch parts 54 branching from trunk part 44 and associated with second expandable stent 26 and third expandable stent 26. In such a way, any two stents of 24, 26 and 32 are associated at a desired distance but with virtually no other limitations as to the relative orientation of the two stents. Analogously to the discussed hereinabove, since tubular parts 44 and 54 of stent connectors 82 and 84 are disposed on the outside of stents 24, 26 and 32, tubular parts 44 and 54 are held in place by tension and the attendant friction. The tubular parts expand together with a respective stent during expansion thereof. In FIG. 6A, the two branch volumes (substantially defined by second expandable stent 26 and third expandable stent 32) are in fluid communication with the trunk volume (substantially defined by first expandable stent 24) through an end of first expandable stent 24 and therefore of the trunk volume. In FIG. 6B, the two branch volumes (substantially defined by second expandable stent 26 and third expandable stent 32) are in fluid communication with the trunk volume (substantially defined by first expandable stent 24) through side openings 45 in first expandable stent 24 and therefore of the trunk volume.

In FIG. 6C is depicted a stent assembly 81 of the present invention comprising a stent connector 85. Stent assembly 81 include three expandable stents, a first expandable stent 24 that is destined for deployment in a trunk vessel upstream of a bifurcation point 16, a second expandable stent 26 destined for deployment in a trunk vessel downstream of a bifurcation point 16, and a third expandable stent 32 that is destined for deployment in a branch vessel. Stent connector 85 is substantially similar to stent connector 66 depicted in FIG. 5A and includes only two tubular parts, a tubular trunk part 44 associated with first expandable stent 24 and second expandable stent 26, and a tubular branch part 54 branching from trunk part 44 and associated with third expandable stent 26. In such a way, any two stents of 24, 26 and 32 are associated at a desired distance but with virtually no other limitations as to the relative orientation. Analogously to the discussed hereinabove, since tubular parts 44 and 54 of stent connector 85 are disposed on the outside of stents 24, 26 and 32, tubular parts 44 and 54 are held in place by tension and the attendant friction. The tubular parts expand together with a respective stent during expansion thereof. In FIG. 6C, the branch volume (substantially defined by third expandable stent 32) is in fluid communication with the trunk volume (substantially defined by first expandable stent 24, second expandable stent 26 and tubular trunk part 44) through the gap between the upstream end of first expandable stent 24 and the downstream end of second expandable stent 26 and therefore of the trunk volume.

It is important to note that stent connectors 66, 68, 70, 72, 82, 84 and 85 depicted in FIGS. 5A, 5B, 5C, 5D, 6A, 6B and 6C are all substantially vessels comprising walls made of a flexible material provided with at least three ports. Stent connectors 66, 68, 70 and 72 are all vessels with three ports where a respective first expandable stent 24 is functionally associated with a first and a second of the three ports while a respective second expandable stent 26 is functionally associated with a third of the three ports. Further, each respective first expandable stent 24 of stent assemblies 58, 60, 62 and 64 includes a side opening 45 that is also functionally associated with the third of the three ports. Stent connectors 82 and 85 are vessels with three ports where a first of three ports is functionally associated with first expandable stent 24, a second of three ports is functionally associated with second expandable stent 26 and a third of three ports is functionally associated with third expandable stent 32. Stent connector 84 is a vessel with four ports where a first and second of four ports are functionally associated with first expandable stent 24, a third of four ports is functionally associated with second expandable stent 26 and a fourth of four ports is functionally associated with third expandable stent 32.

In stent connectors 66, 68, 70 and 72, two ports (associated with first expandable stent 24) and walls of the connector therebetween define a volume that is a trunk part 44 of the stent connector the volume substantially overlapping the trunk volume defined by the bore of a respective first expandable stent 24. Together with the respective first expandable stent 24, trunk part 44 of a stent connector defines a trunk implant, that is the part of the respective stent assembly destined for deployment in a trunk vessel.

In stent connectors 66, 68, 70 and 72 a third port (associated with second expandable stent 26) is in communication with the trunk volume through tubular branch part 54 of the respective stent connector. Together with the respective second expandable stent 26, tubular branch part 54 of a stent connector defines a branch implant, that is the part of the respective stent assembly destined for deployment in the lumen of a branch vessel.

In stent connector 85 of FIG. 6C, two ports and walls of the connector therebetween define a volume that is a trunk part 44 of the stent connector, the volume substantially overlapping the trunk volume defined by the bores of first expandable stent 24 and second expandable stent 26. Together with first expandable stent 24 and second expandable stent 26, trunk part 44 of stent connector 85 defines a trunk implant, that is the part of the respective stent assembly destined for deployment in a trunk vessel. In stent connector 85 a third port (associated with third expandable stent 32) is in communication with the trunk volume through tubular branch part 54. Together with third expandable stent 32, tubular branch part 54 of stent connector 85 defines a branch implant, that is the part of stent assembly 81 destined for deployment in the lumen of a branch vessel.

Stent assemblies of the present invention such as 58, 60, 62, 64, 78 and 80 and 81 are superior to prior art stent assemblies and, for certain uses, to stent assemblies of the present invention such as 22, 30, 34, 36, 46 and 48 due to the nature of the respective stent connector. In these cases, the stent connectors are vessels comprising walls that, when deployed in a bifurcated bodily vessel, support the vicinity of the bifurcation point of the bodily vessel. Such support prevents the collapse of the bodily vessel in the vicinity of the bifurcation point. Further, if during stent deployment and expansion the bodily vessel walls in the vicinity of the bifurcation become weakened, torn or otherwise damaged the respective stent connector acts to contain fluids flowing through the bodily vessel and prevents the fluids breaking through the thus damaged bodily vessel.

As depicted above, depending on the embodiment, tubular trunk parts 44 and tubular branch parts 54 of stent connectors of the present invention are disposed either on the outside of a respective stent or inside the bore of a stent.

Advantages of disposing tubular trunk parts 44 and tubular branch parts 54 inside the bore of stents include providing a smooth lumen wall allowing unrestricted and non-turbulent flow of fluids through the stent connector. Further, such an arrangement is useful for deployment in weakened or damaged vessels as the force applied by fluids flowing through such a stent connector to the tubular part is largely dissipated by the stents.

Advantages of disposing tubular trunk parts 44 and tubular branch parts 54 on the outside of stents include secure and even expansion, avoiding the use of sutures or other methods of securing the stent connector to the stents and allows for efficient relining of damaged vessel walls. Further, such an arrangement encases the area where any two stents meet, preventing pinching of bodily parts between the stents.

In FIGS. 3B, 4A, 4B, 5A, 5B, 5C and 5D first expandable stents 24 of stent assemblies 36, 46, 48, 58, 60, 62 and 64 are provided with a side opening 45. In FIG. 6B, first expandable stent 24 is provided with two side openings 45 (depicted in phantom). Side openings 45 and respective second expandable stents 26 are so positioned that the branch volume (substantially defined by second expandable stent 26) is in fluid communication with the trunk volume (substantially defined by first expandable stent 24) through side opening 45 and therefore from a side of the trunk volume. The use of a stent connector of the present invention ensures that a stent assembly is very bendable allowing changes in relative orientation of the component stents both in and out-of-plane, in a very wide range of divergence angles. At the same time, the use of a stent connector of the present invention ensures that a given branch volume is always in unobstructed fluid communication with a trunk volume through a side opening 45 so that flow therethrough is unimpeded and not turbulent.

As noted hereinabove, it is preferable that the material from which a stent connector of the present invention is fashioned be flexible. In embodiments of the present invention it is also preferable that the stent connector be substantially elastic so as to better retain a desired shape during manipulation and deployment.

In the art stents with a jacket made of a material that is porous and permeable so as to allow cells to grow into and through the material, ultimately leading to the jacket becoming an integral part of the vessel in which deployed are known, see for example U.S. Pat. No. 5,723,004. In embodiments of the present invention, the material from which a stent connector is fashioned is porous and/or permeable so as to allow tissue in-growth. A disadvantage of such permeability is that smooth muscle cells are known to grow in a disorganized and ultimately restenotic fashion through porous and permeable membranes.

In the art stents with a jacket made of a material that is substantially impervious to tissue proliferation therethrough are known, see for example PCT Patent Application PCT/IB98/01459 published as WO 99/15105 of the inventor. Such imperviousness is useful in preventing tissue buildup on and through the material and prevents the migration of smooth muscle cells. In embodiments of the present invention, the material from which a stent connector is fashioned is substantially impervious to tissue proliferation therethrough. Such embodiments are useful for providing a treated vessel with a smooth lining that is substantially impervious to restenosis.

In the art stents with a jacket made of a material that is impermeable to fluids so as to form a sealed vessel and thus avoid extravasation of fluids through the material are known. In embodiments of the present invention, the material from which a stent connector is made is impermeable to fluids.

Useful materials from which to fashion a stent connector of the present invention include synthetic or polymeric material including but not limited to polytetrafluoroethylene, urethane, elastomer, polyamide (e.g., Nylon) and polyester (e.g., Dacron).

Useful materials from which to fashion a stent connector of the present invention are also biological tissue including but not limited to autologous tissue, heterologous tissue, venous tissue, arterial tissue, serous tissue, pleura, peritoneum, pericardium and aortic leaflet. Generally suitable tissue types include but are not limited to equine, porcine, bovine or human tissue. It is often preferred that the tissue be thinned, that is after harvesting one or more layers of the harvested tissue are removed, e.g. by scraping, shaving, slicing or skiving (see U.S. Pat. Nos. 6,468,300 and 6,254,627 of the inventor). In order to increase the toughness of the tissue, it is often advantageous to treat the tissue, for example with a glutaraldehyde or a phosphate solution, in order to cross-link collagen in the tissue.

Serous membranes are made of two strata of tissue. The serous stratum or layer of a serous membrane is a very smooth single layer of flattened, nucleated mesothelial cells united at their edges by a cement substance. The serous cells rest on a basement layer or stratum, a rough, strong fibrous layer. Serous membrane is one material that is strong, elastic and thin enough to be useful in implementing the teachings of the present invention. Thinned serous membrane is even more preferred as noted above and as taught in U.S. Pat. Nos. 6,254,627 and 6,468,300 of the inventor. Not only is thinned serous membrane sufficiently strong, elastic and even thinner than serous membrane, thinned serous membrane also provides little resistance to radial expansion, making thinned serous membrane one of the few materials suitable for use in covering or jacketing self-expanding stents. Thus, in a preferred embodiment, the stent connector is substantially fashioned from a thinned serous membrane where a harvested serous membrane (peritoneum, pericardium or pleural tissue especially porcine, bovine, equine and human serous tissue) has been processed by removal of a layer of at least some of the basement tissue layer (and thus thinned), preferably removal of all the basement tissue, leaving only the serous tissue layer. In an embodiment of the present invention only a part of the basement tissue layer is removed. In an embodiment of the present invention, the thinned serous membrane is substantially serous tissue devoid of basement tissue. In an embodiment of the present invention, the material consists essentially of serous tissue.

When serous membrane or thinned serous membrane is used to implement the teachings of the present invention it is usually preferred to orient the membrane so that the smooth serous strata is facing the fluid flow to reduce turbulent flow.

A stent connector of a bifurcated stent assembly of the present invention is fashioned from one or more parts according to any of the methods with which one skilled in the art is acquainted.

In an embodiment of the present invention, tubular parts of a given stent connector are fashioned from a planar or substantially planar sheet of an appropriate membrane (synthetic or harvested), generally by rolling the sheet (with abutting or overlapping edges) and then fixing the tubular shape using any of the methods known in the art including but not limited to sutures, adhesives, bending members, clamps, glue, hooks, piercing members, staples, other applicable mechanical mean or combinations thereof). A stent connector made up of more than one tubular part is conveniently fashioned by making two separate tubes and conjoining the two tubes (using, for example, sutures, adhesives, bending members, clamps, glue, hooks, piercing members, staples, other applicable mechanical mean or combinations thereof).

In an embodiment of the present invention a stent connector is fashioned including seamless tubular parts. In an embodiment, a seamless tubular part of a stent connector of the present invention is fashioned from synthetic materials by methods including but not limited to, weaving synthetic fibers, molding a polymer or welding the seam of a rolled sheet, for instance using heat or an appropriate solvent. In an embodiment, a seamless tubular part of a stent connector of the present invention is fashioned from a section of harvested tubular biological tissue. In such a case a suitable tubular vessel, such as an appropriately sized autologous, homologous or heterologous artery or vein is identified, harvested, isolated and treated to prepare a seamless tubular stent connector component. Treatments include chemical or biological treatments, for example cross-linking or digestion, to increase flexibility or strength of the material of the tubular stent connector component. Treatments also include mechanical thinning to increase the flexibility and reduce thickness of the material of the stent connector component.

In embodiments of the present invention, a stent connector is made of two different materials. For example, the tubular stent connector wall is fashioned from a thin tube of serous tissue attached to a second tubular stent connector part made of a thicker material, for example, a harvested artery.

In an embodiment of the present invention a bifurcated stent connector is seamless. In an embodiment, a seamless bifurcated stent connector of the present invention is fashioned from synthetic materials by methods including but not limited to, weaving synthetic fibers, molding a polymer or welding separate components, for instance using heat or an appropriate solvent. In a preferred embodiment, a seamless bifurcated stent connector of the present invention is fashioned from a section of a bifurcated biological tissue. In such a case a suitable bifurcated vessel, such as an appropriately sized autologous, homologous or heterologous bifurcated artery or vein is identified, harvested, isolated and treated to prepare a seamless bifurcated stent connector. Treatments include chemical or biological treatments, for example cross-linking or digestion, to increase flexibility or strength of the material of the vessel and ultimately of the thus-fashioned stent connector. Treatments also include mechanical thinning to increase the flexibility and reduce thickness of the material of the vessel.

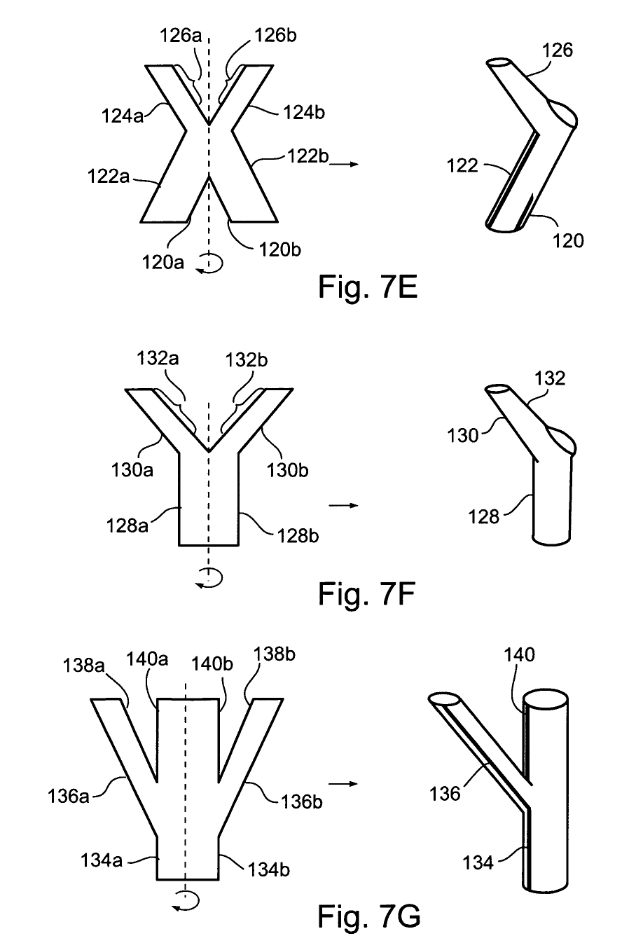

In an embodiment of the present invention, a bifurcated stent connector of the present invention is essentially fashioned from one sheet of planar or substantially planar sheet of an appropriate membrane (synthetic or harvested), rolled up into shape (with abutting or overlapping edges) and then fixed in shape using any of the methods known in the art including but not limited to sutures, adhesives, bending members, clamps, glue, hooks, piercing members, staples, other applicable mechanical mean or combinations thereof). Such an embodiment is preferred over a seamless bifurcated stent connector as the availability of suitable bifurcated vessels for harvesting is limited and limits the materials from which stent connector are fashioned. Such an embodiment is preferred over a bifurcated stent connector fashioned from two or more conjoined tubular components due to the reduced number of seams. Preferred shapes of sheets suitable for use in fashioning a bifurcated stent connector from one sheet of material generally include shapes having one C2 symmetry axis and at least one external angle of no greater than 90° and/or at least 7 sides. In FIGS. 7A-7G are depicted various suitable shapes rolled up and edges attached to fashion bifurcated stent connectors of the present invention.

In FIG. 7A is depicted a cross-shaped sheet, rolled up as depicted by the arrow so that sides 100a and 100b are joined to form seam 100, sides 102a and 102b are joined to form seam 102, sides 104a and 104b are joined to form seam 104 and sides 106a and 106b are joined to form a non-depicted seam to fashion a bifurcated stent connector.

In FIG. 7B is depicted a “T”-shaped sheet, rolled up as depicted by the arrow so that sides 108a and 108b are joined to form seam 108, sides 110a and 110b are joined to form a non-depicted seam and sides 112a and 112b (delimited by the braces) are joined to form a seam 112 to fashion a bifurcated stent connector.

In FIGS. 7C and 7D are depicted “W”-shaped sheets, rolled up as depicted by the arrow so that sides 114a and 114b are joined to form seam 114, sides 116a and 116b are joined to form a non-depicted seam and sides 118a and 118b are joined to form a seam 118 to fashion a bifurcated stent connector.

In FIG. 7E is depicted an “X”-shaped sheet, rolled up as depicted by the arrow so that sides 120a and 120b are joined to form seam 120, sides 122a and 122b are joined to form seam 122, sides 124a and 124b are joined to form a non-depicted seam, and sides 126a and 126b (delimited by the braces) are joined to form a seam 126 to fashion a bifurcated stent connector.

In FIG. 7F is depicted a “Y”-shaped sheet, rolled up as depicted by the arrow so that sides 128a and 128b are joined to form seam 128, sides 130a and 130b are joined to form seam 130 and sides 132a and 132b are joined to form seam 132 to fashion a bifurcated stent connector.

In FIG. 7G is depicted a “Ψ”-shaped sheet, rolled up as depicted by the arrow so that sides 134a and 134b are joined to form seam 134, sides 136a and 136b are joined to form seam 136, sides 138a and 138b are joined to form a non-depicted seam and sides 140a and 140b are joined to form seam 140 to fashion a bifurcated stent connector.

Any suitable method known in the art may be used to conjoin any two components or to attach ends of a single piece of material to fashion a stent connector of the present invention. Such methods include but are not limited to sutures, adhesives, clamps, glue, hooks, piercing members, staples, laser welding other applicable mechanical mean or combinations thereof. In embodiments of the present invention two components or two ends of a single piece of material are overlapped and joined. An overlapping joint is leak resistant and strong due to increased surface area of the seam. In embodiments of the present invention two components or two ends of a single piece of material are abutted and joined. An abutting joint is thinner than an overlapping joint.

Once a stent connector of the present invention of the desired topography is made, assembly of a bifurcated stent assembly is straightforward for one skilled in the art upon perusal of the disclosure herein. Generally, a stent is selected and contacted with an appropriate part of the stent connector. When a stent is surrounded by a substantially tubular part of a stent connector, the stent is generally threaded through the tubular part to the desired extent. As noted hereinabove, a given stent is secured to an appropriate part of a respective stent connector with sutures or by a combination of tension and concomitant friction of a tubular stent connector section surrounding a stent. That said, in embodiments of the present invention a stent is secured to a respective stent connector using any of the methods known in the art, including but not limited to adhesives, bending members, clamps, glue, hooks, piercing members, staples, other applicable mechanical mean or combinations thereof).

One of the design features of a stent connector of the present invention comprising a tubular part is the length of the tubular part relative to a stent with which the tubular part is associated, that is to say, what part of the outer surface of a stent is covered by a respective tubular part (when the tubular part is disposed outside the stent) or what part of the inside surface of a stent is in contact with a respective tubular part (when the tubular part is disposed inside the stent). As noted hereinabove, in embodiments of the present invention a tubular part is substantially as long or even longer than a stent with which that tubular part is associated. In embodiments of the present invention the tubular part is substantially shorter than an associated stent, for example up to 50% of the length of an associated stent, up to 40% of the length of an associated stent, up to 30% of the length of an associated stent or even up to 20% of the length of an associated stent.

Generally the walls of a stent connector of a bifurcated stent assembly of the present invention are as thin as possible yet must be elastic enough and strong enough to be useful, that is to allow navigation and deployment of the bifurcated stent assembly including movement of the component stents of the bifurcated stent assembly without tearing. Clearly, the nature of the material from which a given stent connector is made determines in part the thickness of that stent connector. That said, a stent connector of a bifurcated stent assembly of the present invention preferably has walls that are not thicker than about 0.75 mm, not thicker than about 0.45 mm, not thicker than about 0.25 mm, not thicker than about 0.20 mm, and even not thicker than about 0.05 mm. A useful material from which to fashion a stent connector of a bifurcated stent assembly of the present invention having the appropriate thickness yet also being smooth to reduce the chances of restenosis, being sufficiently strong and flexible is thinned serous membrane as discussed above, especially bovine pericardium as disclosed in PCT Patent application No. PCT/IB98/01459 published as WO 99/15105 of the inventor.

Generally any type of stent known in the art is useful as a component of a bifurcated stent assembly of the present invention. Such stents include but are not limited to stents marketed by affiliates (e.g., Cordis, Centocor) of Johnson & Johnson, Guidant (Indianapolis, Indiana, USA), Medtronic (Minneapolis, Minnesota, USA), Medinol (Tel Aviv, Israel), Cook Inc. (Bloomington, Indiana, USA) and PM Devices Inc. (Richmond, British Columbia, Canada). In embodiments of the stent assembly of the present invention, a first expandable stent, a second expandable stent (and third or more expandable stents if present) are of substantially similar or identical dimensions, especially length, expanded diameter and/or unexpanded diameter.

In embodiments of the present invention a first expandable stent, a second expandable stent (and third or more expandable stents if present) are of substantially different dimensions, especially length, expanded diameter and/or unexpanded diameter. In cases where the first expandable stent, the second expandable stent (and third or more expandable stents if present) are of different dimensions, generally the first expandable stent is larger (especially of larger expanded and/or unexpanded diameter) as it is generally the first expandable stent that is destined to be deployed in a trunk vessel rather than a smaller-bored branch vessel.

In embodiments of the present invention, the diameter (expanded or unexpanded) of the first expandable stent is substantially similar to the respective diameter of the second expandable stent (and third or more expandable stents if present).

In embodiments of the present invention, the diameter (expanded or unexpanded) of the first expandable stent is larger than the respective diameter of the second expandable stent (and third or more expandable stents if present).

In embodiments of a bifurcated stent assembly of the present invention a first expandable stent has a length of up to about 80 mm, up to about 65 mm, or even up to about 50 mm.

In embodiments of a bifurcated stent assembly of the present invention a first expandable stent has a length of greater than about 5 mm, greater than about 7 mm, or even greater than about 10 mm.

In embodiments of a bifurcated stent assembly of the present invention a second expandable stent has a length of up to about 80 mm, up to about 65 mm, or even up to about 50 mm.

In embodiments of a bifurcated stent assembly of the present invention a second expandable stent has a length of greater than about 5 mm, greater than about 7 mm, or even greater than about 10 mm.

Two important parameters used when selecting a stent for use are the expanded and unexpanded diameters of the stent.

Generally it is important that the unexpanded diameter of a stent be as small as possible to ease navigation through the bodily lumen to the deployment location. That said, the unexpanded diameter must be large enough to allow threading of the stent onto a deployment catheter and, if necessary, a stent-expanding device such as a stent-expanding balloon. Although there may be some variation in the unexpanded diameter of even two identical stents depending on how the two stents are used, herein by unexpanded diameter is intended the outer diameter of an expandable stent when crimped to the greatest extent onto a delivery catheter for deployment.

In embodiments of a bifurcated stent assembly of the present invention a first expandable stent has an unexpanded diameter as defined above of at least about 0.5 mm, at least about 1 mm, and even at least about 2 mm. In embodiments of a bifurcated stent assembly of the present invention a second expandable stent (or third or more expandable stents if present) has an unexpanded diameter as defined above of at least about 0.5 mm, at least about 1 mm, and even at least about 2 mm.

Generally, any given stent has a wide range of expanded diameters larger than a respective unexpanded diameter. The expanded diameter of a stent subsequent to deployment is determined by the user of the stent according to medical criteria including the natural size of the lumen of the vessel in which the stent is deployed. That said, self-expanding stents are characterized by a specific maximal expansion that is the maximal diameter of the stent when the stent is free from externally applied forces. Most not self-expanding stents are also characterized by a maximal expansion that is the greatest extent to which the stent is expandable without comprising the structural integrity thereof.

In embodiments of a bifurcated stent assembly of the present invention a first expandable stent has a maximal expanded diameter as defined above of up to about 30 mm, up to about 8 mm, up to about 6 mm, and even up to about 5 mm.

In embodiments of a bifurcated stent assembly of the present invention a second expandable stent (and third or more expandable stents if present) has a maximal expanded diameter as defined above of up to about 30 mm up, to about 8 mm, up to about 6 mm, and even up to about 5 mm.

Generally, a first expandable stent and a second expandable stent of a bifurcated stent assembly of the present invention are relatively close together to ensure maximal support of a treated vessel when deployed. Thus, in embodiments of the present invention the distance between the first expandable stent and the second expandable stent is no greater than about four, no greater than about three, no greater than about two, no greater than about one and even no greater than about half of an unexpanded diameter of the second expandable stent.

It is known in the art to deploy a stent provided with a stent jacket. The stent jacket is generally a tubular membrane placed on the outer surface of the stent although internal stent jackets, substantially tubular membranes held within the bore of the stent, are known (see, for example, U.S. Pat. Nos. 6,254,627 and 6,699,277). The stent jacket provides a smooth lumen for the treated vessel, reduces turbulent flow through the treated vessel, and provides structural reinforcement. In embodiments of the present invention, one or more of the component stents of a bifurcated stent assembly are jacketed. For example, any of the different stent jackets known to one skilled in the art are useful for jacketing one, some or all component stents of a stent assembly of the present invention.

It is known in the art to deploy a coated stent. Many different coatings are known in the art, for example, anti-thrombogenic coatings, anti-angiogenic coatings, anti-coagulant coatings and active pharmaceutical ingredient delivering coatings. Any of the different stent coatings known to one skilled in the art are useful for coating one, some or all component stents of a stent assembly of the present invention.

Deployment of a stent assembly of the present invention is preferably performed according to the methods known in the art, for example as described in U.S. Pat. No. 5,723,004 or PCT Patent application PCT/IB98/00496 published as WO 99/15103 of the inventor. Deployment of a stent assembly 58 depicted in FIG. 5A into a vessel 10 as depicted in FIG. 1A is discussed in detail hereinbelow, where first expandable stent 24 and second expandable stent 26 are non-self expanding.

In brief, according to one method of deploying a stent assembly of the present invention, two guidewires are navigated through the body, up through a trunk vessel to be treated, and past bifurcation point 16, a first guidewire in trunk vessel 12 and a second guidewire into branch vessel 14. Thereafter, stent assembly 58 in the unexpanded state is mounted on a delivery system including one delivery catheter for each stent of stent assembly 58, preferably including two balloon catheters for each stent of stent assembly 58, or a balloon catheter for first expandable (trunk) stent 24 and a transfer catheter for second expandable (branch) stent 26. The delivery catheter on which first expandable stent 24 is mounted is positioned over the first guidewire and the delivery catheter on which second expandable stent 26 is mounted is positioned over the second guidewire. The two delivery catheters are advanced along the respective guidewires and thus navigated through the body to the proper location in vessel 10, first expandable stent 24 between lines A-A and B-B and across bifurcation point 16 and second expandable stent 26 into branch part 14. Stent connector 66 is placed snugly against the bifurcation as stent connector 66 prevents any possibility of pushing stent assembly 58 too far and damaging vessel 10. Further, stent connector 66 allows each component stent, 24 and 26 to bend and independently move relative to the other, without leading to any distortion, bending or buckling in the other stent. Second expandable stent 26 is pushed into branch part 14 until resistance of stent connector 66 is felt. In such a way it is ensured that second expandable stent 26 is not too close to bifurcation point 16 and does not protrude into trunk vessel 12. At the same time, stent connector 66 prevents second expandable stent 26 from being pushed too far into branch part 14. Once properly located first expandable stent 24 and second expandable stent 26 are expanded, whether simultaneously or serially, each to an expanded state of a desired size. Expansion of a stent generally occurs by positioning an inflation device (such as the balloon of a balloon catheter) inside the bore of a respective stent, inflating the balloon to a desired extent so as to expand the stent to a desired extent, and susequently deflating the balloon for withdrawal from the body.

In an embodiment where a delivery device includes a balloon catheter for each stent, the balloon is generally already properly located within the bore of a respective stent. First expandable stent 24 and second expandable stent 26 are either simultaneously or serially expanded to a desired extent by inflation of the respective balloons. The balloons are deflated and the delivery catheters withdrawn from the body.

In an embodiment where a delivery device already includes a balloon catheter on which first expandable stent 24 is mounted and a transfer catheter (without a balloon) on which second expandable stent 26 is mounted, the balloon is generally already properly located within the bore of first expandable stent 24. In a first step, the balloon is inflated to expand first expandable stent 24 to a desired extent and deflated. In a second step, the transfer catheter is withdrawn and a replacement balloon catheter advanced over the second guidewire until the balloon thereon is properly positioned inside the bore of second expandable stent 26. The balloon of the replacement balloon catheter is inflated to expand second expandable stent 26 to a desired extent and deflated. The two catheters are withdrawn from the body.

Deployment of other stent assemblies of the present invention and in vessels such as 10 depicted in FIG. 1A or 18 depicted in FIG. 1B, or when first expandable stent 24 or second expandable stent 26 or both are self-expanding is understood by one skilled in the art upon perusal of the description herein.

Although described with respect to treating bifurcated vessels of the cardiovascular system, and especially bifurcated arteries, the teachings of the present invention are generally applicable to many different cardiovascular and non-cardiovascular applications. Specific cardiovascular applications include but are not limited to the deployment of a bifurcated stent of the present invention in ectatic arteries and ectatic arteries containing an obstructive lesion, aneurismatic arteries, saphenous vein grafts and native arteries, coronary perforation, coronary fistula, ostial coronary lesions, aortic abdominal aneurysm and other aneurismatic peripheral arteries, transjugular intrahepatic portal shunt, percutaneous transluminal angioplasty, fistula closing and neuro interventions (such as aneurysms and arterial-venous malformations), small vessel intraluminal grafting, and ostial renal artery lesions. Additional non-cardiovascular applications include but are not limited to urological, gastroenterological, respiratory and neurological applications.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for bendably associating a first expandable stent with a second expandable stent for deployment in a bifurcated vessel comprising using a flexible membrane as a stent connector to connect the second expandable stent to the first expandable stent so that the bore of the first expandable stent defines an elongated trunk volume and the bore of the second expandable stent defines an elongated branch volume branching from said trunk volume wherein the two stents are substantially devoid of direct physical association.

2. The method of claim 1, wherein the two stents are devoid of mutual physical contact.

3. The bifurcated stent assembly of claim 1, wherein said stent connector comprises at least one filament.

4. The method of claim 1, wherein at least part of said stent connector is substantially tubular.

5. The method of claim 4, wherein said stent connector includes a substantially tubular trunk part functionally associated with the first expandable stent.

6. The method of claim 5, wherein said trunk part of said stent connector surrounds at least part of the first expandable stent.

7. The method of claim 5, wherein at least part of said trunk part of said stent connector is disposed within the bore of said first expandable stent.

8. The method of claim 4, wherein said stent connector includes a substantially tubular branch part functionally associated with the second expandable stent.

9. The method of claim 5, wherein said branch part of said stent connector surrounds at least part of the second expandable stent.

10. The method of claim 5, wherein at least part of said branch part of said stent connector is disposed within the bore of said second expandable stent.

11. The method of claim 4, wherein said stent connector includes a first substantially tubular trunk part functionally associated with the first expandable stent and a second substantially tubular branch part functionally associated with the second expandable stent, said branch part branching from said trunk part.

12. The method of claim 1, wherein said branch volume is in fluid communication with said trunk volume from a side of said trunk volume.

13. The method of claim 1, wherein said branch volume is in fluid communication with said trunk volume from an end of said trunk volume.

14. A bifurcated stent assembly, comprising:
a) a flexible membrane as a stent connector;
b) a first expandable stent functionally associated with said stent connector, wherein the bore of said first expandable stent defines an elongated trunk volume; and
c) a second expandable stent functionally associated with said stent connector, wherein the bore of said second expandable stent defines an elongated branch volume branching from said trunk volume wherein said stents are substantially devoid of direct physical association.

15. The bifurcated stent assembly of claim 14, wherein said stents are devoid of mutual physical contact.

16. The bifurcated stent assembly of claim 14, wherein said stent connector comprises at least one filament.

17. The bifurcated stent assembly of claim 14, wherein said branch volume is in fluid communication with said trunk volume from a side of said trunk volume.

18. The bifurcated stent assembly of claim 17, wherein said first expandable stent is provided with a side opening through which said branch volume is in fluid communication with said trunk volume.

19. The bifuircated stent assembly of claim 14, wherein said branch volume is in fluid communication with said trunk volume from an end of said trunk volume.

20. The bifurcated stent assembly of claim 14, wherein a said stent is secured to said stent connector by at least one member of the group consisting of sutures, adhesives, bending members, clamps, glue, hooks, piercing members and staples.

21. The bifurcated stent assembly of claim 14, wherein at least part of said stent connector is substantially tubular.

22. The bifurcated stent assembly of claim 21, wherein said stent connector includes a substantially tubular trunk part functionally associated with said first expandable stent.

23. The bifurcated stent assembly of claim 22, wherein said first substantially tubular part of said stent connector surrounds at least part of said first expandable stent.

24. The bifurcated stent assembly of claim 23, wherein said first expandable stent is substantially entirely contained within said stent connector.

25. The bifurcated stent assembly of claim 24, wherein said first expandable stent is substantially entirely contained within said trunk part of said stent connector.

26. The bifurcated stent assembly of claim 23, wherein at least one end of said first expandable stent emerges from said stent connector.

27. The bifurcated stent assembly of claim 26, wherein two ends of said first expandable stent emerge from said stent connector.

28. The bifurcated stent assembly of claim 22, wherein at least part of said trunk part of said stent connector is disposed within the bore of said first expandable stent.

29. The bifurcated stent assembly of claim 28, wherein said first expandable stent is substantially entirely outside said stent connector.

30. The bifurcated stent assembly of claim 28, wherein said trunk part of said stent connector is substantially entirely disposed within the bore of said first expandable stent.

31. The bifurcated stent assembly of claim 28, wherein a first end of said trunk part of said stent connector emerges from a first end of said first expandable stent.

32. The bifurcated stent assembly of claim 31, wherein said first end of said trunk part of said stent connector emerges from a first end of said first expandable stent and is folded thereover.

33. The bifurcated stent assembly of claim 31, wherein a second end of said trunk part of said stent connector emerges from a second end of said first expandable stent.

34. The bifurcated stent assembly of claim 33, wherein said second end of said trunk part of said stent connector emerges from a second end of said first expandable stent and is folded thereover.

35. The bifurcated stent assembly of claim 21, wherein said stent connector includes a substantially tubular branch part functionally associated with said second expandable stent.

36. The bifurcated stent assembly stent assembly of claim 35, wherein said branch part of said stent connector surrounds an end of said second expandable stent.

37. The bifurcated stent assembly of claim 36, wherein said second expandable stent is substantially entirely contained within said stent connector.

38. The bifurcated stent assembly of claim 37, wherein said second expandable stent is substantially entirely contained within said branch part of said stent connector.

39. The bifurcated stent assembly of claim 36, wherein an end of said second expandable stent emerges from said branch part of said stent connector.

40. The bifurcated stent assembly of claim 35, wherein at least part of said branch part of said stent connector is disposed within the bore of said second expandable stent.

41. The bifurcated stent assembly of claim 40, wherein said second expandable stent is substantially entirely outside said stent connector.

42. The bifurcated stent assembly of claim 40, wherein said branch part of said stent connector is substantially entirely disposed within the bore of said second expandable stent.

43. The bifurcated stent assembly of claim 40, wherein an end of said branch part of said stent connector emerges from an end of said second expandable stent.

44. The bifurcated stent assembly of claim 43, wherein said end of said branch part of said stent connector emerging from said end of said second expandable stent is folded thereover.

45. The bifurcated stent assembly of claim 21, wherein said stent connector includes a first substantially tubular trunk part functionally associated with said first expandable stent and a second substantially tubular branch part functionally associated with said second expandable stent, said branch part branching from said trunk part.

46. The bifurcated stent assembly of claim 45, wherein said first expandable stent is provided with a side opening, said stent connector is at least partially disposed within the bore of said first expandable stent so that said branch part emerges from said side opening.

47. The bifurcated stent assembly of claim 14, further comprising:
d) a third expandable stent functionally associated with said stent connector, wherein the bore of said third expandable stent defines an elongated second branch volume branching from said trunk volume.

48. The bifurcated stent assembly of claim 47, wherein said second branch volume is in fluid communication with said trunk volume from a side of said trunk volume.

49. The bifurcated stent assembly of claim 47, wherein said second branch volume is in fluid communication with said trunk volume from an end of said trunk volume.

50. The bifurcated stent assembly of claim 47, wherein said stent connector includes a substantially tubular branch part functionally associated with said third expandable stent.

51. The bifurcated stent assembly of claim 14, wherein said stent connector is substantially a vessel comprising walls of a flexible material and at least three ports, a first port, a second port and a third port, and, wherein said first expandable stent is functionally associated with said first port and said second expandable stent is functionally associated with said third port.

52. The bifurcated stent assembly of claim 51, further comprising a third expandable stent functionally associated with said second port.

53. The bifurcated stent assembly of claim 51, wherein said first port, said second port and walls of said stent connector therebetween define a trunk part of said stent connector enclosing a volume that substantially overlaps said trunk volume.

54. The bifurcated stent assembly of claim 51, wherein said stent connector comprises exactly three ports.

55. The bifurcated stent assembly of claim 51, wherein said first expandable stent is functionally associated with said second port.

56. The bifurcated stent assembly of claim 51, wherein said first expandable stent is provided with a side opening.

57. The bifurcated stent assembly of claim 56, wherein said side opening of said first expandable stent is functionally associated with said third port of said stent connector.

58. The bifurcated stent assembly of claim 56, wherein the bore of said second expandable stent is in fluid communication with the bore of said first expandable stent through said side opening.

59. The bifurcated stent assembly of claim 53, wherein said first port, said second port, walls of said stent connector therebetween together with said first expandable stent substantially define at least part of a trunk implant.

60. The bifurcated stent assembly of claim 53, wherein said third port is in fluid communication with said trunk volume through a substantially tubular branch of said stent connector.

61. The bifurcated stent assembly of claim 60, wherein said third port, walls of said substantially tubular branch of said stent connector together with said second expandable stent substantially define at least part of a branch implant.

62. The bifurcated stent assembly of claim 61, wherein at least part of said tubular branch is contained within the bore of said second expandable stent.

63. The bifurcated stent assembly of claim 62, wherein said tubular branch is substantially entirely contained within the bore of said second expandable stent.

64. The bifurcated stent assembly of claim 62, wherein a part of said tubular branch emerges from an end of said second expandable stent.

65. The bifurcated stent assembly of claim 64, wherein a part of said tubular branch emerges from an end of said second expandable stent and is folded thereover.

66. The bifurcated stent assembly of claim 14, wherein said flexible membrane is substantially elastic.

67. The bifurcated stent assembly of claim 14, wherein said flexible membrane is substantially impermeable to fluids.

68. The bifurcated stent assembly of claim 14, wherein said flexible membrane is substantially impervious to tissue proliferation therethrough.

69. The bifurcated stent assembly of claim 14, wherein said flexible membrane is permeable.

70. The bifurcated stent assembly of claim 14, wherein said flexible membrane is substantially fashioned from a material selected from the group consisting of synthetic materials, polymeric materials, polytetrafluoroethylene, urethane, elastomer, polyamide and polyester.

71. The bifurcated stent assembly of claim 14, wherein said flexible membrane is substantially fashioned from a material selected from the group consisting of biological tissue, autologous tissue, heterologous tissue, venous tissue, arterial tissue, serous tissue, pleura, peritoneum, pericardium and aortic leaflet.

72. The bifurcated stent assembly of claim 71, wherein said material substantially comprises a section of a bifurcated biological tissue.

73. The bifurcated stent assembly of claim 71, wherein said material substantially comprises a section of a bifurcated blood vessel.

74. The bifurcated stent assembly of claim 71, wherein said material is harvested from a source selected from the group consisting of human sources and animal sources.

75. The bifurcated stent assembly of claim 74, wherein said material is harvested from a source selected from the group consisting of equine, porcine, bovine and human sources.

76. The bifurcated stent assembly of claim 71, wherein said material is thinned.
